(19) United States
(12) Patent Application Publication
(10) Pub. No.: US 2010/0221146 A1
(43) Pub. Date: Sep. 2, 2010

(54) METHOD OF ASEPTICALLY TREATING, FORMING, FILLING, AND SEALING FLEXIBLE BAG PACKAGES IN A BAG PACKAGING MACHINE

(76) Inventor: Thomas MATHEYKA, Eppstein (DE)

Correspondence Address: NILS H. LJUNGMAN & ASSOCIATES, P. O. BOX 130, GREENSBURG, PA, 15601-0130, US

(21) Appl. No.: 12/548,181

(22) Filed: Aug. 26, 2009

Related U.S. Application Data
(63) Continuation-in-part of application No. PCT/EP2008/000244, filed on Jan. 15, 2008.

(30) Foreign Application Priority Data
Feb. 27, 2007 (DE) 10 2007 009 798.2

(51) Int. Cl. A61L 2/025; B06B 1/00; B65B 55/04; B65B 43/04

(52) U.S. Cl. 422/20; 422/127; 53/426; 53/455; 53/562; 53/167

(57) ABSTRACT

A method of aseptically treating, forming, filling, and sealing flexible bag packages in a bag packaging machine. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/EP2008/000244, filed on Jan. 15, 2008, which claims priority from Federal Republic of Germany Patent Application No. 10 2007 009 798.2, filed on Feb. 27, 2007. International Patent Application No. PCT/EP2008/000244 was pending as of the filing date of this application. The United States was an elected state in International Patent Application No. PCT/EP2008/000244.

### BACKGROUND

1. Technical Field

The present application relates to a method of aseptically treating, forming, filling, and sealing flexible bag packages to minimize the presence of living contaminants on packaging material in a bag packaging machine.

2. Background Information

Background information is for informational purposes only and does not necessarily admit that subsequently mentioned information and publications are prior art.

Among other things, including for the bottling or packaging of beverages, foodstuffs or other products, such as perishable products, for example, in bags, one of the practical requirements that has recently acquired increased importance is the extension of the shelf life of the packaged product. This requirement applies possibly for aseptic bottling methods (cold or hot bottling) for products, the bottling process for which omits a thermal sterilization after the filling process, and possibly, among other things, reduced manufacturing losses caused by rejections and an improved shelf life. One requirement or desired for aseptic bottling processes that are performed in sterile or clean rooms is the prevention of contamination of the packaging by microorganisms.

In some methods, when the packaging material is taken from a supply roll and the roll is located outside the sterile or clean room, to prevent, restrict, and/or minimize contaminating the sterility of the clean room when an empty supply roll is replaced by a new supply roll, it is necessary and/or desired to sterilize the packaging material when it is fed into the sterile or clean room and therefore before it is fed to the packaging machine which is located in said sterile or clean room.

Some methods describe the sterilization of packaging material with peroxyacetic acid, with hydrogen peroxide (H₂O₂), and/or with UV radiation. These methods have significant disadvantages. For example, methods that use peroxyacetic acid or hydrogen peroxide are expensive and also time-consuming, simply because a separate activation time must be or should be reserved for the sterilization. If an effective sterilization is to be achieved with these methods, it is therefore possible to operate at very low production rates or throughputs. An additional disadvantage is that the media peroxyacetic acid or hydrogen peroxide which are used with these processes are potentially hazardous to the health of the operating and plant personnel, not to mention the fact that a certain amount of residue of the media that have been used generally remains on the packaging material.

The irradiation of the packaging material with UV radiation or UV light is cheaper and is basically also safe, provided that the equipment used is properly designed and operated. However, very approximate sterilization effects or sterilization rates can be achieved by treatment with UV radiation.

The prior art also describes methods to kill microorganisms by ultrasound on surfaces of products, such as, for example, on the surfaces of postal shipments, and in one possible embodiment with the use of an ultrasound device which is used in contact with the respective surface, or with the use of an ultrasound source which is located at a distance from the respective surface to be treated.

Finally, some methods and devices for cleaning the cavity of an object, such as the lumen of a medical instrument, for example, involves introducing a transmission fluid into this interior space with the ultrasound energy of an ultrasound source.

The present application relates to a method for the sterilization of packaging material, for example for the sterilization of films or similarly flexible wrapping materials, and to a device for the sterilization of packaging material, in one possible embodiment of films or similar flexible wrapping materials.

### OBJECT OR OBJECTS

An object of the present application is a method which makes it possible to achieve a highly effective sterilization of packaging material which does not use peroxyacetic acid, hydrogen peroxide or similar disinfecting liquid media.

### SUMMARY

To accomplish this object, the present application teaches a method for the sterilization of packaging material, in one possible embodiment for the sterilization of films or similarly flexible wrapping materials, comprising the irradiation of the packaging material with mechanical ultrasound energy to kill microorganisms by exposure to the action of mechanical and/or thermal energy. Another object of the present application is a device for the performance of the method.

The present application teaches that the sterilization of the packaging material, which is usually supplied in a strip format, is accomplished with ultrasound energy, and in one possible embodiment so that any or most microorganisms that are on the surface of the packaging material are killed and/or rendered harmless by mechanical as well as thermal destruction of their cell walls by protein coagulation and ultimately by denaturing of their DNA.

The product that is packaged using the packaging material that has been treated in the manner described above, such as, for example, the product (in one possible embodiment including beverages or foods) that is filled into bags that are manufactured from the packaging material, is reliably protected from spoiling, i.e. the quality and shelf life of the product are significantly improved compared to the previously described methods.

During the sterilization of the packaging material with ultrasound treatment, no toxic or hazardous byproducts are created, and no residue of treatment media remains on the sterilized packaging material, as is the case in the other methods that use peroxyacetic acid or hydrogen peroxide.

In at least one possible embodiment of the present application, the at least one ultrasound system used for the ultrasound treatment is self-cleaning, and the properties of the packaging material are not altered, or not essentially altered, by the ultrasound treatment. It is also possible to use the heating of the packaging material that is generated during the ultrasound treatment for a subsequent thermal process. The ultrasound treatment takes place, for example, at an ultrasound frequency in the range between 20,000 Hertz and one GigaHertz, but in one possible embodiment in the range between 20,000 Hertz and 35,000 Hertz.

For the ultrasound treatment, one or more ultrasound units are provided on the path of movement of the strip-format packaging material, and are then activated simultaneously or timed and chronologically delayed with respect to one another in a timed sequence, or for the emission of the ultrasound energy onto the packaging material, although in the event of a timed activation, the ultrasound units are activated so that there is a temporary overlapping of the activation periods, to thereby effectively prevent, restrict, and/or minimize gaps in the sterilization of the packaging material.

The individual ultrasound unit comprises, for example, an electrically operated generator which in its capacity as an electromechanical converter generates a mechanical ultrasound oscillation from a mechanical resonance unit and/or an amplifier to amplify the oscillation, as well as an ultrasound heat or a sonotrode, i.e. a piece of metal or tool that is exposed to ultrasonic vibration, and then transfers this vibratory energy in an element to apply. For the ultrasound treatment, the packaging material is pulled under mechanical tension across this sonotrode or its acoustic pressure zone and is thereby irradiated to kill any or most germs or microorganisms that may be present with the ultrasound energy. The packaging material can thereby be guided directly over the sonotrode, i.e. without any space in between and in contact with the sonotrode. It is also possible to guide the packaging material at some distance from the sonotrode, i.e. so that it does not contact the sonotrode. The type of guidance of the packaging material, either in contact with the sonotrode or at some distance from it, can be selected as a function of the parameters of the specific application.

By modifying the mechanical tension of the packaging material, the angle of incidence of the sonotrode and/or by the selection of the sonotrode geometry, the intensity of the ultrasound treatment can be adapted to the requirements of the specific application, in one possible embodiment to achieve the desired sterilization rate or the desired sterilization degree without any or with little damage or destruction of the packaging material.

Because the transmission of the ultrasonic oscillations in air is relatively poor, it may also be necessary or desired to uniformly apply moisture or liquid onto the packaging material, and in one possible embodiment before and/or during the ultrasound treatment. The moisture or liquid can thereby be applied by condensation. In this manner, the introduction of the ultrasound energy into the packaging material can be increased by a multiple. The thermal action of the ultrasound treatment then also results in an accelerated drying of the packaging material that has been moistened for the treatment.

The above-discussed embodiments of the present invention will be described further herein below. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

Developments of the present application are described according to the present application. The present application is explained in greater detail below with reference to the possible embodiment which is illustrated in the accompanying drawings, in which:

FIG. 1 is a schematic illustration of a device for the sterilization of a strip-format packaging material in the form of a packaging film;

FIG. 2 is a schematic illustration of the device illustrated in FIG. 1 in use with a packaging machine;

FIG. 3 is a simplified plan view of the packaging material extracted from a supply roll or film roll, together with an ultrasound system with a plurality of ultrasound units;

FIG. 4 is a schematic illustration of the device illustrated in FIG. 1 in use with a packaging machine;

FIG. 5 is a schematic illustration of the device illustrated in FIG. 1 in use with a packaging machine; and

FIG. 6 shows a block diagram of the treatment arrangement of the present application in use with a packaging machine, filling machine, and a sealing machine.

### DESCRIPTION OF EMBODIMENT OR EMBODIMENTS

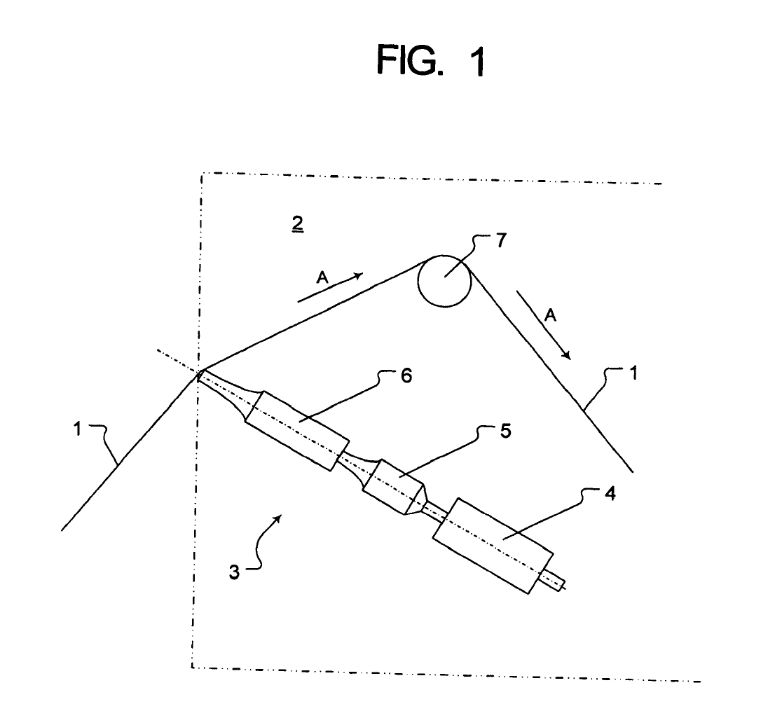

FIG. 1 shows a strip-format packaging material in the form of a packaging film (e.g. shrink film) which is fed into a clean room 2 and thereby passes through an ultrasound unit 3 which forms a lock at the entrance to the clean room, in which the packaging material 1 is treated with mechanically generated ultrasound energy, and in one possible embodiment for the sterilization or killing of any or most germs or microorganisms that may be present on the packaging material 1. In the illustrated possible embodiment, the ultrasound unit 3 comprises an ultrasound generator or converter 4 which is powered at the line voltage and generates the mechanical ultrasonic oscillation at a frequency which can, for example, be in the range between 20,000 and 35,000 Hertz, an amplifier or booster 5 which amplifies the mechanical ultrasound oscillation and an ultrasound heat, which is also called a sonotrode 6, which forms a contact surface that extends across the entire strip width of the packaging material 1. The packaging material 1 is guided across the sonotrode 6 under tension, whereby the sonotrode 6 can be pressed against the packaging material 1, for example.

The packaging material 1 is pulled by means of a motor-driven advance mechanism 7 across the sonotrode 6 (direction of transport A). Like the ultrasound unit 3, the motor-driven advance mechanism 7 is also located in the clean room 2, which is flushed by a laminar flow of sterile air, for example.

The intensity of the ultrasound treatment can be adapted to meet individual and specific requirements by modifying the mechanical tension of the packaging material 1 and/or by changing the angle of incidence of the sonotrode 6 and/or by the selection of the geometry of the sonotrode and/or by applying liquid to the packaging material 1 before and/or during the passage over the sonotrode 6. The liquid that influences and/or promotes the ultrasound treatment and/or of a gaseous medium that influences or promotes the ultrasound treatment can be applied, for example, by means of corresponding nozzles which are located in the direction of transport A upstream of the sonotrode 6 and are directed toward the side that is in contact with the contact surface of the sonotrode 6, i.e. in the illustration in FIG. 1 toward the underside of the packaging material 1, and/or over the sonotrode 6 itself.

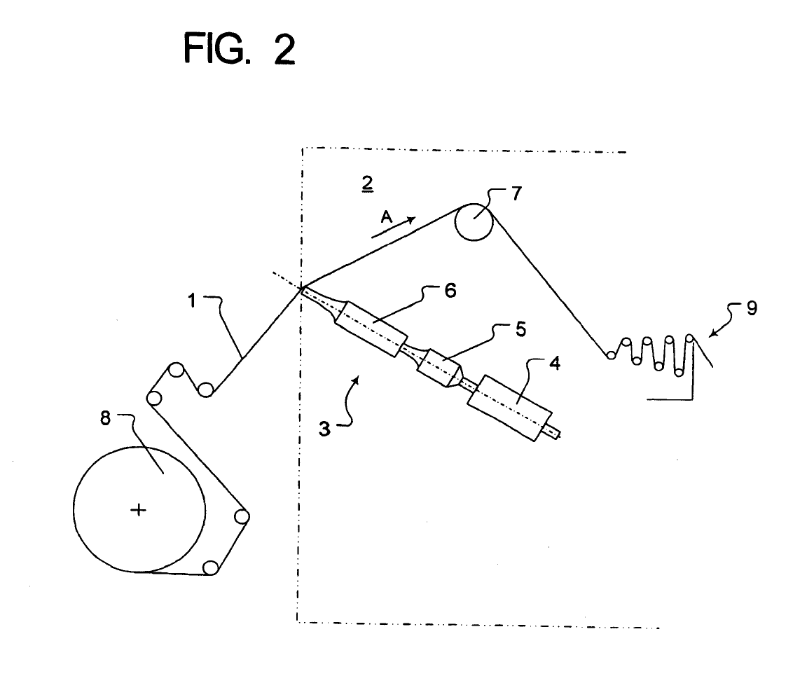

FIG. 2 once again shows the device for sterilization of the packaging material 1, which is formed essentially by the ultrasound unit, together with additional components of a plant which includes a packaging machine and is otherwise illustrated partly and very schematically, and in which packages such as bags, for example, are fabricated from the packaging material 1 and a product is aseptically packaged in these packages.

FIG. 2 shows a supply roll 8 located outside the clean room 2, from which the strip-format packaging material 1 is pulled off (in direction of transport A) via various pulleys, and under tension, across the sonotrode 6 of the ultrasound unit 3 which is in turn located at the inlet to the clean room 2 or in the lock located at the inlet to the clean room 2.

After passing the sonotrode 6 and the motor-driven advance mechanism, the packaging material travels via a buffer 9 formed by a plurality of rollers to the actual packaging machine, which is not shown in FIG. 2 and is located in the clean room 2.

The packaging material 1 is in one possible embodiment continuously or substantially continuously pulled across the sonotrode 6 by means of the motor-driven advance mechanism. The packaging material 1 is pulled off at a speed which ensures or promotes the desired sterilizing effect or the desired sterilization degree taking into consideration the ultrasound energy which is delivered to the packaging material 1 with the ultrasound unit 3.

Downstream from the buffer 9, the packaging material 1 is delivered to the packaging machine by a discontinuous advance movement.

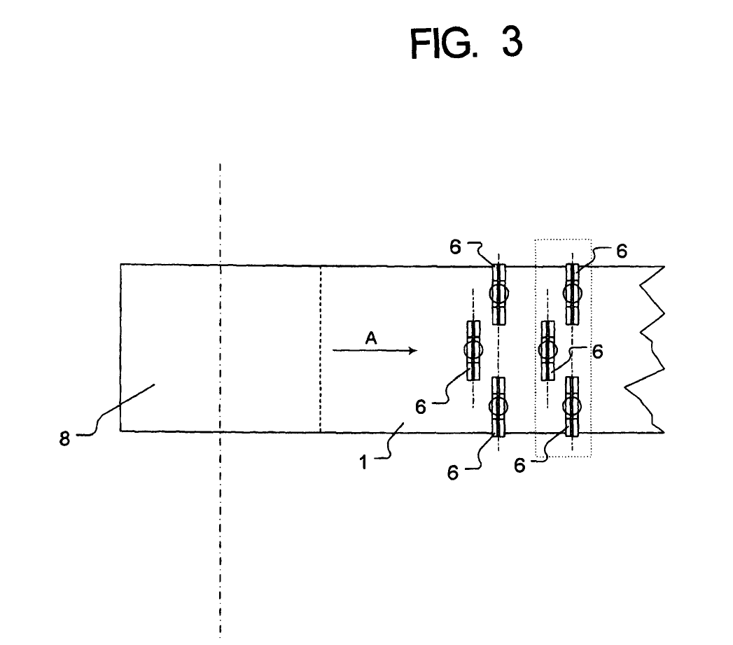

FIG. 3 again shows a plan view of the packaging material 1 pulled off the supply roll 8 in the direction of transport A. In this embodiment, a total of 6 ultrasound units 3 are provided for the sterilization of this material, and in one possible embodiment each with a sonotrode 6. The sonotrodes 6, over which the packaging material is once again pulled under mechanical tension, are arranged in a group so that they treat the complete surface of the packaging material, and in the illustrated embodiment in four rows which are oriented at a right angle to the direction of transport A, whereby the first and second rows in the direction of transport A each have, in the center of the strip of the packaging material 1, a sonotrode 6 which is oriented with its longitudinal dimension oriented transversely across the direction of transport A, and the second and fourth rows each have two sonotrodes 6 oriented with their longitudinal dimension oriented transversely across the direction of transport A in the peripheral areas of the strip of packaging material. The grouping is further selected so that the sonotrodes 6 of neighboring rows overlap. The individual ultrasound units 3 are operated simultaneously or substantially simultaneously, for example, or the ultrasound units 3 can be operated in alternation, in one possible embodiment to prevent, restrict, and/or minimize an overloading of these units and/or of the packaging material 1 by excessive ultrasound energy. In any case, the ultrasound units are operated so that gaps in the sterilization of the packaging material 1 are prevented, restricted, and/or minimized. In at least one possible embodiment, the activation of the ultrasound units 3 overlaps at every point of their operation.

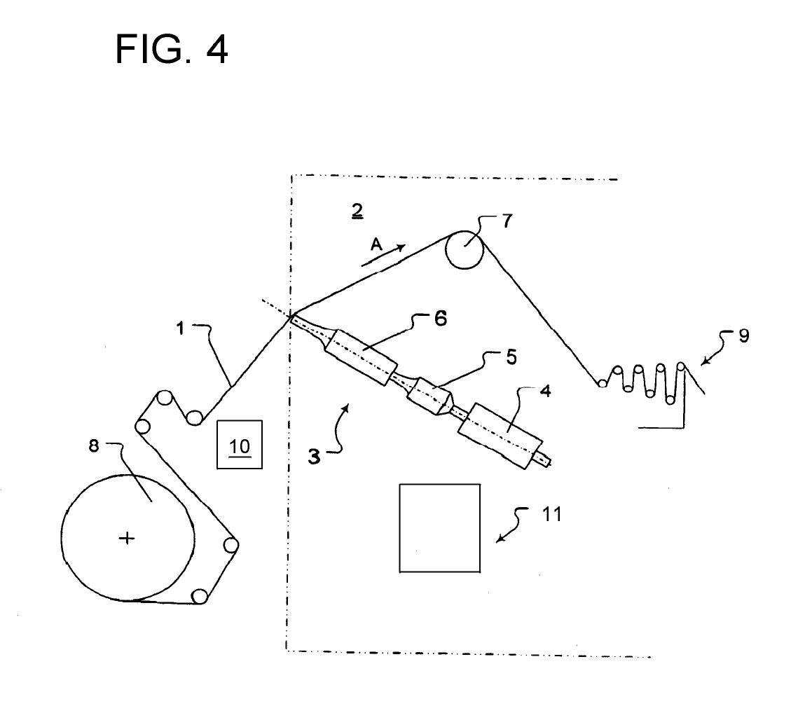

FIG. 4 also shows the device for sterilization of the packaging material 1, which is formed essentially by the ultrasound unit, together with additional components of a plant which includes a packaging machine and is otherwise illustrated partly and very schematically, and in which packages such as bags, for example, are fabricated from the packaging material 1 and a product is aseptically packaged in these packages.

FIG. 4 shows a supply roll 8 located outside the clean room 2, from which the strip-format packaging material 1 is pulled off (in direction of transport A) via various pulleys, and under tension, across the sonotrode 6 of the ultrasound unit 3 which is in turn located at the inlet to the clean room 2 or in the lock located at the inlet to the clean room 2.

After passing the sonotrode 6 and the motor-driven advance mechanism, the packaging material travels via a buffer 9 formed by a plurality of rollers to the actual packaging machine, which is not shown in FIG. 2 and is located in the clean room 2.

The packaging material 1 is in one possible embodiment continuously or substantially continuously pulled across the sonotrode 6 by means of the motor-driven advance mechanism. The packaging material 1 is pulled off at a speed which ensures or promotes the desired sterilizing effect or the desired sterilization degree taking into consideration the ultrasound energy which is delivered to the packaging material 1 with the ultrasound unit 3.

Downstream from the buffer 9, the packaging material 1 is delivered to the packaging machine by a discontinuous advance movement.

Also seen in FIG. 4 is a nozzle arrangement 10, which is configured to apply a liquid or gaseous medium to the packaging material 1. The liquid and/or gaseous medium may influence or promote the ultrasound or ultrasonic treatment of the packaging material 1. The nozzle arrangement is disposed to apply the liquid or gaseous medium to the side of the packaging material 1 which is nearer the sonotrode 6 and/or will come into contact with the sonotrode 6.

Another feature of the present application seen in FIG. 4 is an adjustment arrangement 11. This arrangement or controlling arrangement 11 may be configured to change the angle of incidence of the sonotrode 6 and/or the selection of the sonotrode geometry. The adjustment arrangement 11 may also be configured to change the intensity of the ultrasound or ultrasonic treatment of the packaging material 1, depending upon the desired treatment of the packaging material 1, for example, to achieve the desired or predetermined sterilization rate of the desired or predetermined sterilization degree without any or with minimal damage or deconstruction of the packaging material 1. The control unit 11 may be configured to control or manage the functions of the ultrasound unit 3. For example, the control unit 11 may be configured to control the generator or converter 4, control the amplifier 5, and control the positioning or angle of the sonotrode 6. In other possible embodiments of the present application, the control arrangement 11 may be configured to control other functions or parameters of the ultrasonic or ultrasound unit 3.

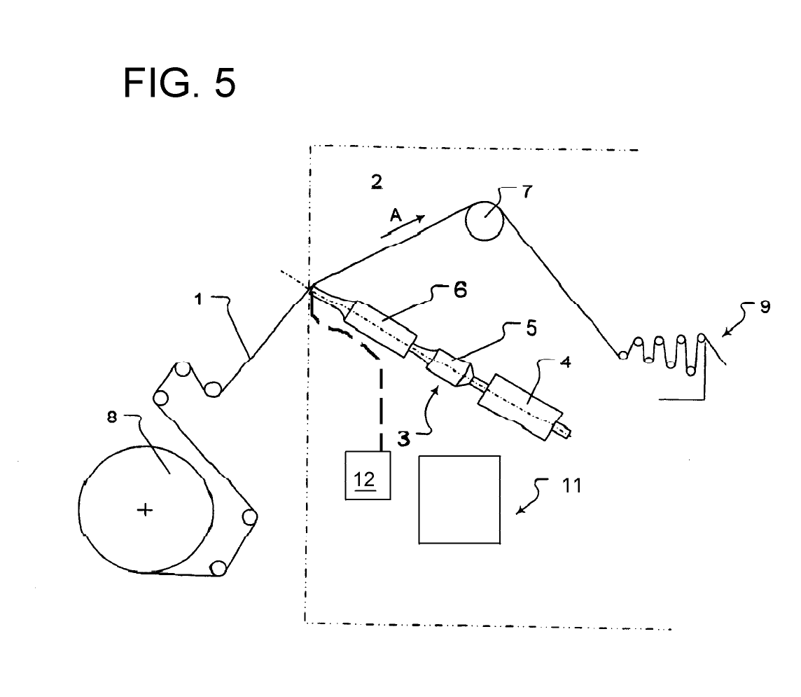

FIG. 5 also shows the device for sterilization of the packaging material 1, which is formed essentially by the ultrasound unit, together with additional components of a plant which includes a packaging machine and is otherwise illustrated partly and very schematically, and in which packages such as bags, for example, are fabricated from the packaging material 1 and a product is aseptically packaged in these packages.

Also seen in FIG. 5 is a supply arrangement 12, which may be configured to supply liquid and/or gaseous medium to the sonotrode 6. The sonotrode 6 of the ultrasonic or ultrasound device 3 is configured to supply the medium to the packaging material 1 for the treatment.

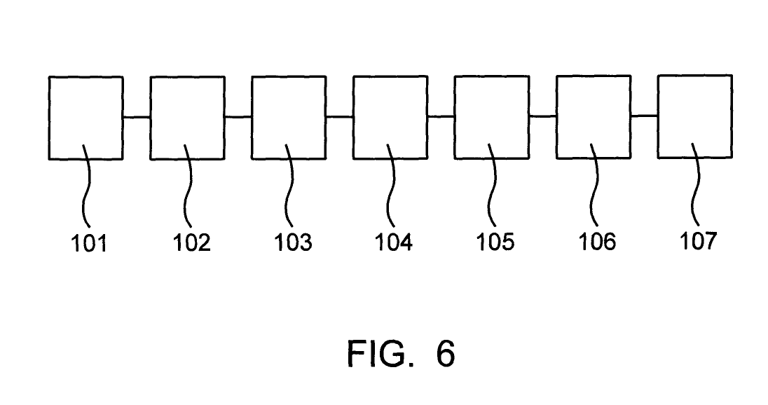

FIG. 6 is a block diagram of the ultrasound device 3 of the present application. A supply 101 of packaging material may be fed into the treating arrangement 102. The treating arrangement 102 may comprise the ultrasound unit 3 of the present application. The treated packaging material is then fed into the separator or divider 103. The packaging material may be then fed into the forming arrangement 104, and the packaging material is formed into bag packages. The formed bag packages may then be transported to the filling arrangement 105, where the bags are filled with a material. The filled bags are then transported to the closing arrangement 106, where the bags are closed. The filled, closed bags are then transported to a packaging arrangement 107. The packaging arrangement 107 then packages the filled, closed bags into shipping containers for distribution.

The present application was described above with reference to possible embodiments. It goes without saying that numerous modifications and variations can be made without thereby going beyond the teaching of the present application.

Method for the sterilization of packaging material, in one possible embodiment for the sterilization of films or similar flexible wrapping materials, in which the packaging material is irradiated with mechanical ultrasound energy to kill microorganisms by mechanical energy and/or thermal treatment.

One feature or aspect of an embodiment is believed at the time of the filing of this patent application to possibly reside broadly in the method for the sterilization of packaging material, in one possible embodiment for the sterilization of films or similarly flexible wrapping materials, comprising the irradiation of the packaging material 1 with mechanical ultrasound energy to kill microorganisms by exposure to the action of mechanical and/or thermal energy.

Another feature or aspect of an embodiment is believed at the time of the filing of this patent application to possibly reside broadly in the method, wherein the packaging material 1 is moved under mechanical tension over at least at least one ultrasound head 6 of at least one ultrasound unit 3.

Yet another feature or aspect of an embodiment is believed at the time of the filing of this patent application to possibly reside broadly in the method, wherein the ultrasound head 6 forms at least one strip-like contact area that extends transversely across the path of the strip-format packaging material 1 for the packaging material.

Still another feature or aspect of an embodiment is believed at the time of the filing of this patent application to possibly reside broadly in the method, wherein the packaging material is moved continuously across the ultrasound head 6.

A further feature or aspect of an embodiment is believed at the time of the filing of this patent application to possibly reside broadly in the method, wherein the intensity of the ultrasound treatment can be modified by changing the angle of incidence of the ultrasound oscillations or the ultrasound energy onto the packaging material 1 and/or by the selection of the geometry of the at least one ultrasound head 6 and/or by the application force of the packaging material against the at least one ultrasound head 6.

Another feature or aspect of an embodiment is believed at the time of the filing of this patent application to possibly reside broadly in the method, wherein the angle of incidence of the ultrasound head 6 is adjustable.

Yet another feature or aspect of an embodiment is believed at the time of the filing of this patent application to possibly reside broadly in the method, wherein a medium which promotes the ultrasound treatment is applied to the packaging material 1 before and/or during the ultrasound treatment.

Still another feature or aspect of an embodiment is believed at the time of the filing of this patent application to possibly reside broadly in the method, wherein the medium is a liquid and/or a gas.

A further feature or aspect of an embodiment is believed at the time of the filing of this patent application to possibly reside broadly in the method, wherein the medium that promotes the ultrasound treatment is a medium that improves the transmission of the ultrasound energy from the at least one ultrasound head 6 to the packaging material 1.

Another feature or aspect of an embodiment is believed at the time of the filing of this patent application to possibly reside broadly in the method, wherein the medium that promotes the ultrasound treatment is applied by means of the at least one ultrasound head 6 to the packaging material 1.

Yet another feature or aspect of an embodiment is believed at the time of the filing of this patent application to possibly reside broadly in the method, wherein the ultrasound treatment is performed by a plurality of ultrasound heads 6 or ultrasound units 3 and in one possible embodiment simultaneously or chronologically delayed with respect to one another.

Still another feature or aspect of an embodiment is believed at the time of the filing of this patent application to possibly reside broadly in the method, wherein the ultrasound treatment is performed at a frequency in the range between approximately 20,000 Hertz and one gigahertz.

A further feature or aspect of an embodiment is believed at the time of the filing of this patent application to possibly reside broadly in the method, wherein the ultrasound treatment is performed at a frequency in the range between approximately 20,000 and 35,000 Hertz.

One feature or aspect of an embodiment is believed at the time of the filing of this patent application to possibly reside broadly in the method, wherein the ultrasound energy is controlled as a function of the speed at which the packaging material 1 passes the at least one ultrasound head 6.

Another feature or aspect of an embodiment is believed at the time of the filing of this patent application to possibly reside broadly in a device for the sterilization of packaging material 1, in one possible embodiment of films or similar flexible wrapping materials, comprising at least one ultrasound system with at least one ultrasound unit 3 with an ultrasound head 6, over which the strip-format packaging material 1 is guided.

Yet another feature or aspect of an embodiment is believed at the time of the filing of this patent application to possibly reside broadly in the device, wherein the at least one ultrasound 6 forms at least one strip-shaped contact surface for the packaging material 1 which runs transversely across the path of the packaging material 1.

Still another feature or aspect of an embodiment is believed at the time of the filing of this patent application to possibly reside broadly in the device, wherein the tension with which the packaging material 1 is in contact against the ultrasound head 6 or its contact area is adjustable.

One feature or aspect of an embodiment is believed at the time of the filing of this patent application to possibly reside broadly in a method for the sterilization of packaging material, in one possible embodiment for the sterilization of films or similarly flexible wrapping materials by the irradiation of the packaging material 1 with mechanical ultrasound energy to kill microorganisms by exposure to the action of mechanical and/or thermal energy, wherein, the packaging material 1 is moved under mechanical tension over at least at least one ultrasound head 6 of at least one ultrasound unit 3.

Another feature or aspect of an embodiment is believed at the time of the filing of this patent application to possibly reside broadly in the method, wherein the packaging material 1 is guided across at least one strip-like contact area that extends transversely across the path of the strip-format packaging material 1 and forms the ultrasound head 6 for the packaging material.

Yet another feature or aspect of an embodiment is believed at the time of the filing of this patent application to possibly reside broadly in the method, wherein the packaging material is moved continuously or substantially continuously across the ultrasound head 6.

Still another feature or aspect of an embodiment is believed at the time of the filing of this patent application to possibly reside broadly in the method, wherein the intensity of the ultrasound treatment can be modified by changing the angle of incidence of the ultrasound oscillations or the ultrasound energy onto the packaging material 1 and/or by the selection of the geometry of the at least one ultrasound head 6 and/or by the application force of the packaging material against the at least one ultrasound head 6.

A further feature or aspect of an embodiment is believed at the time of the filing of this patent application to possibly reside broadly in the method, wherein the angle of incidence of the ultrasound head 6 is adjustable.

Another feature or aspect of an embodiment is believed at the time of the filing of this patent application to possibly reside broadly in the method, wherein a medium which promotes the ultrasound treatment is applied to the packaging material 1 before and/or during the ultrasound treatment.

Yet another feature or aspect of an embodiment is believed at the time of the filing of this patent application to possibly reside broadly in the method, wherein the medium is a liquid and/or a gas.

Still another feature or aspect of an embodiment is believed at the time of the filing of this patent application to possibly reside broadly in the method, wherein the medium that promotes the ultrasound treatment is a medium that improves the transmission of the ultrasound energy from the at least one ultrasound head 6 to the packaging material 1.

A further feature or aspect of an embodiment is believed at the time of the filing of this patent application to possibly reside broadly in the method, wherein the medium that promotes the ultrasound treatment is applied by means of the at least one ultrasound head 6 to the packaging material 1.

Another feature or aspect of an embodiment is believed at the time of the filing of this patent application to possibly reside broadly in the method, wherein the ultrasound treatment is performed by a plurality of ultrasound heads 6 or ultrasound units 3 and in one possible embodiment simultaneously or chronologically delayed with respect to one another.

Yet another feature or aspect of an embodiment is believed at the time of the filing of this patent application to possibly reside broadly in the method, wherein the ultrasound treatment is performed at a frequency in the range between approximately 20,000 Hertz and one GigaHertz.

Still another feature or aspect of an embodiment is believed at the time of the filing of this patent application to possibly reside broadly in the method, wherein the ultrasound treatment is performed at a frequency in the range between approximately 20,000 and 35,000 Hertz.

A further feature or aspect of an embodiment is believed at the time of the filing of this patent application to possibly reside broadly in the method, wherein the ultrasound energy is controlled as a function of the speed at which the packaging material 1 passes the at least one ultrasound head 6.

One feature or aspect of an embodiment is believed at the time of the filing of this patent application to possibly reside broadly in a device for the performance of the method according to the present application, comprising at least one ultrasound system with at least one ultrasound unit 3 with an ultrasound head 6, over which the strip-format packaging material 1 is guided.

Another feature or aspect of an embodiment is believed at the time of the filing of this patent application to possibly reside broadly in the device, wherein the at least one ultrasound 6 forms at least one strip-shaped contact surface for the packaging material 1 which runs transversely across the path of the packaging material 1.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner. All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

All of the patents, patent applications or patent publications, which were cited in the International Search Report dated Apr. 16, 2008, and/or cited elsewhere are hereby incorporated by reference as if set forth in their entirety herein as follows: US 2004/028552, having the title “GAS CONTACT ULTRASOUND GERMICIDE AND THERAPEUTIC TREATMENT,” published on Feb. 12, 2004; U.S. Pat. No. 4,591,485, having the title “METHOD AND APPARATUS FOR SONICATING ARTICLES,” published on May 27, 1986; US 2006/021642, having the title “APPARATUS AND METHOD FOR DELIVERING ACOUSTIC ENERGY THROUGH A LIQUID STREAM TO A TARGET OBJECT FOR DISRUPTIVE SURFACE CLEANING OR TREATING EFFECTS,” published on Feb. 2, 2006; JP 2002/321813, having the following English translation of the Japanese title “BELT CONVEYOR WASHING MACHINE,” published on Nov. 8, 2002; and U.S. Pat. No. 4,494,357, having the title “STERILIZATION OF PACKAGING MATERIAL,” published on Jan. 22, 1985.

All of the patents, patent applications or patent publications, which were cited in the German Office Action dated Oct. 12, 2007, and/or cited elsewhere are hereby incorporated by reference as if set forth in their entirety herein as follows: WO2006/073645, having the title “PROCESS FOR THE DESTRUCTION OF MICROORGANISMS ON A PRODUCT USING ULTRASONIC ENERGY,”published on Jul. 13, 2006; and DE 10 2005 055 291, having the following English translation of the German title “Method and device for ultrasonically cleaning hollow bodies,” published on Jun. 6, 2007.

Some examples of ultrasonic amplifiers, which may possibly be utilized or adapted for use in at least one possible embodiment of the present application, may possibly be found in the following U.S. Pat. No. 7,396,336, having the title “Switched resonant ultrasonic 659 power amplifier system,” published on Jul. 8, 2008; U.S. Pat. No. 5,435,186, having the title “Ultrasonic parametric amplifier,” published on Jul. 25, 1995; U.S. Pat. No. 4,164,143, having the title “Ultrasonic amplifier detector,” published on Aug. 14, 1979; U.S. Pat. No. 7,306,506, having the title “In-situ chemical-mechanical planarization pad metrology using ultrasonic imaging,” published on Dec. 11, 2007; and U.S. Pat. No. 7,191,698, having the title “System and technique for ultrasonic determination of degree of cooking,” published on Mar. 20, 2007.

Some examples of sonotrodes, which may possibly be utilized or adapted for use in at least one possible embodiment of the present application, may possibly be found in the following U.S. Pat. No. 7,344,620, having the title “Ultrasonic sonotrode,” published on Mar. 18, 2008; U.S. Pat. No. 6,776,323, having the title “Sonotrode for ultrasound welding of plastics,” published on Aug. 17, 2004; U.S. Pat. No. 6,691,909, having the title “Sonotrode for ultrasonic welding apparatus,” published on Feb. 17, 2004; and U.S. Pat. No. 5,426,341, having the title “Sonotrode for ultrasonic machining device,” published on Jun. 20, 1995.

Some examples of ultrasonic generators or ultrasound generators or transducers, which may possibly be utilized or adapted for use in at least one possible embodiment of the present application, may possibly be found in the following U.S. Pat. No. 7,521,023, having the title “Apparatus and methods for controlling sonic treatment,” published on Apr. 21, 2009; U.S. Pat. No. 7,497,119, having the title “Methods and systems for using resonant acousto-EM energy and acousto-EM signatures to disrupt biological structures,” published on Mar. 3, 2009; U.S. Pat. No. 7,416,535, having the title “Neoplastic cell destruction device and method utilizing low frequency sound waves to disrupt or displace cellular materials,” published on Aug. 26, 2008; and U.S. Pat. No. 7,025,735, having the title “Ultrasonic apparatus for the treatment of septic wounds,” published on Apr. 11, 2006.

One example of an ultrasound generator which may possibly be utilized or adapted for use in at least one possible embodiment of the present application may include the Chattanooga Ultrasound Generator, model Intelect 230-P, sold by Miga Solutions, having the address 3500 Holly Lane N, Suite 40, Plymouth, Minn. 55447. Another example of an ultrasound generator which may possibly be utilized or adapted for use in at least one possible embodiment of the present application may include the Ultrasound Rife Frequency Generator, designed and produced by physicist Gary Wade, sold by Mega Brain Power, having the address Harvard Square, Cambridge, Mass. 02238. A third example of an ultrasonic generator which may possibly be utilized or adapted for use in at least one possible embodiment of the present application may include the Ultrasonic welding generator, of the DIGITAL SE series, sold by Sirius Electric, having the address Via Mastronardi 6/b, 27029 Vigevano (PV), Italy.

Some examples of nozzles or nozzle arrangements for spraying liquid and gaseous mediums, which may possibly be utilized or adapted for use in at least one possible embodiment of the present application, may possibly be found in the following U.S. Pat. No. 6,808,681, having the title “Method of sterilizing a package material,” published on Oct. 26, 2004; U.S. Pat. No. 5,163,487, having the title “Method and apparatus for dispensing a liquid into containers in an aseptic or sterile manner,” published on Nov. 17, 1992; U.S. Pat. No. 7,010,900, having the title “BEVERAGE BOTTLING PLANT FOR FILLING BOTTLES WITH A LIQUID BEVERAGE FILLING MATERIAL, AND A CLEANING DEVICE FOR CLEANING BOTTLES IN A BEVERAGE BOTTLING PLANT,” published on Mar. 14, 2006; U.S. Pat. No. 6,042,026 issued to Buehler, II on Mar. 28, 2000; U.S. Pat. No. 6,394,366 issued to Adams on May 28, 2002; U.S. Pat. No. 6,402,062 issued to Bendig et al. on Jun. 11, 2002; U.S. Pat. No. 6,616,072 issued to Harata et al. on Sep. 9, 2003; U.S. Pat. No. 6,666,386 issued to Huang on Dec. 23, 2003; and No. 6,681,498 issued to Steffan on Jan. 27, 2004. U.S. patent application Ser. No. 12/341,564 filed on Dec. 22, 2008, having inventor Thomas MATHEYKA, Attorney Docket No. NHL-HOL-206, and its corresponding Federal Republic of Germany Patent Application No. DE 10 2006 028 796.7, and International Patent Application No. PCT/EP2007/05299, filed on Jun. 20, 2007, having WIPO Publication No. WO 2007/147566 and inventor Thomas MATHEYKA are hereby incorporated by reference as if set forth in their entirety herein.

U.S. patent application Ser. No. 12/342,440 filed on Dec. 23, 2008, having inventor Thomas MATHEYKA, Attorney Docket No. NHL-HOL-207, and its corresponding Federal Republic of Germany Patent Application No. 10 2006 030 255.9, filed on Jul. 30, 2006, and International Patent Application No. PCT/EP2007/008402, filed on Jun. 20, 2007, having WIPO Publication No. WO 2008/000376 and inventor Thomas MATHEYKA are hereby incorporated by reference as if set forth in their entirety herein.

Some examples of apparatuses for opening and disposing bags and of gripping arrangements that may possibly be utilized or adapted for use in at least one possible embodiment may possibly be found in the following a U.S. Pat. No. 4,108,300, having the title “BAG PACKING APPARATUS,” published on Aug. 2, 1978; and No. 6,276,117, having the title “ADJUSTABLE POUCH CARRIER FOR DIFFERENT SIZED POUCHES AND PACKAGING MACHINE HAVING AN ADJUSTABLE POUCH CARRIER,” published on Aug. 21, 2001.

Some examples of apparatuses for closing arrangements that may possibly be utilized or adapted for use in at least one possible embodiment may possibly be found in US Patent Application No. 2007/0101684, having the title “DEVICE FOR THE PRODUCTION OF TUBULAR BAGS,” published on May 10, 2007.

Some examples of sheet materials, which may possibly be utilized or adapted for use in at least one possible embodiment of the present application, may possibly be found in the following U.S. Pat. No. 7,550,198, having the title “Multilayer sealable film 756 having a temperature-resistant layer therein,” published on Jun. 23, 2009; U.S. Pat. No. 7,351,942, having the title “Insulating microwave interactive packaging,” published on Apr. 1, 2008; U.S. Pat. No. 7,281,360, having the title “Thermal laminates and laminating method of food packaging films,” published on Oct. 16, 2007; U.S. Pat. No. 6,932,592, having the title “Metallocene-produced very low density polyethylenes,” published on Aug. 23, 2005; U.S. Pat. No. 6,372,361, having the title “Coating for paper products,” published on Apr. 16, 2002; and No. 5,405,663, having the title “Microwave package laminate with extrusion bonded susceptor,” published on Apr. 11, 1995.

Some examples of methods and apparatus for forming bags out of sheet material, which may possibly be utilized or adapted for use in at least one possible embodiment of the present application, may possibly be found in the following U.S. Pat. No. 7,553,269, having the title of “Method of manufacturing a bag,” published on Jun. 30, 2009; U.S. Pat. No. 7,549,269, having the title “Method of forming a bag having a reclosable seal,” published on Jun. 23, 2009; U.S. Pat. No. 7,546,722, having the title “Vertical filling-packaging machine and method of manufacturing a packaging bag,” published on Jun. 16, 2009; U.S. Pat. No. 7,396,163, having the title “Paper and plastic bags flexible packages and other containers with re-closable device and method of making the same,” published on Jul. 8, 2008; U.S. Pat. No. 7,033,077, having the title “Sealable bags made of plastics or other materials and method of making plastic sheeting for manufacturing sealable bags,” published on Apr. 25, 2006; U.S. Pat. No. 7,361,872, having the title “Variable serving size insulated packaging,” published on Apr. 22, 2008; U.S. Pat. No. 5,773,801, having the title “Microwave cooking construction for popping corn,” published on Jun. 30, 1998; U.S. Pat. No. 7,067,781, having the title “Single ply paper product, method for manufacturing, and article,” published on Jun. 27, 2006; and No. 4,348,851, having the title “Packing machine for the production of filled sealed bags,” published on Sep. 14, 1982.

Some examples of control systems which measure operating parameters and learn therefrom that may possibly be utilized or possibly adapted for use in at least one possible embodiment of the present application may possibly be found in the following U.S. Pat. No. 4,655,188 issued to Tomisawa et al. on Apr. 7, 1987; No. 5,191,272 issued to Torii et al. on Mar. 2, 1993; U.S. Pat. No. 5,223,820, issued to Sutterlin et al. on Jun. 29, 1993; and No. 5,770,934 issued to Theile on Jun. 23, 1998.

Some examples of apparatuses and methods for packaging filled, sealed bags into boxes, which may possibly be utilized or adapted for use in at least one possible embodiment of the present application, may possibly be found in the following U.S. Pat. No. 7,361,850, having the title “Method and apparatus for counting items in a container,” published in Apr. 22, 2008; U.S. Pat. No. 6,988,350, having the title “Packaging system,” published in Jan. 24, 2006; and No. 5,251,422, having the title “Potato chip package vertical packaging machine,” published on Oct. 12, 1993.

The patents, patent applications, and patent publications listed above in the preceding fifteen paragraphs are herein incorporated by reference as if set forth in their entirety. The purpose of incorporating U.S. patents, Foreign patents, publications, etc. is solely to provide additional information relating to technical features of one or more embodiments, which information may not be completely disclosed in the wording in the pages of this application.

Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

The corresponding foreign and international patent publication applications, namely, Federal Republic of Germany Patent Application No. 10 2007 009 798.2, filed on Feb. 27, 2007, having inventor Thomas MATHEYKA, and DE-OS 10 2007 009 798.2 and DE-PS 10 2007 009 798.2, and International Application No. PCT/EP2008/000244, filed on Jan. 15, 2008, having WIPO Publication No. WO 2008/104246 and inventor Thomas MATHEYKA, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

The purpose of incorporating the corresponding foreign equivalent patent application(s), that is, PCT/EP2008/000244 and German Patent Application 10 2007 009 798.2, is solely for the purpose of providing a basis of correction of any wording in the pages of the present application, which may have been mistranslated or misinterpreted by the translator. Words relating to opinions and judgments of the author and not directly relating to the technical details of the description of the embodiments therein are not to be incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned word in this sentence, when not used to describe technical features of one or more embodiments, are not generally considered to be incorporated by reference herein.

Statements made in the original foreign patent applications PCT/EP2008/000244 and DE 10 2007 009 798.2 from which this patent application claims priority which do not have to do with the correction of the translation in this patent application are not to be included in this patent application in the incorporation by reference.

Any statements about admissions of prior art in the original foreign patent applications PCT/EP2008/000244 and DE 10 2007 009 798.2 are not to be included in this patent application in the incorporation by reference, since the laws relating to prior art in non-U.S. Patent Offices and courts may be substantially different from the Patent Laws of the United States.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):

A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.

Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

### AT LEAST PARTIAL NOMENCLATURE

1 Packaging material or film
2 Clean room
3 Ultrasound unit
4 Converter or ultrasonic oscillation generator
5 Amplifier or booster
6 Sonotrode
7 Motor-driven advance mechanism
8 Supply roll
9 Buffer

A Direction of transport

What is claimed is:

1. A method of aseptically treating, forming, filling, and sealing flexible bag packages in a packaging machine, said method comprising the steps of:
unrolling a roll of packaging material and feeding said packaging material to an opening in an aseptic clean room;
feeding said packaging material under tension through said opening and simultaneously treating said packaging material as said packaging material moves through said opening;
cutting a predetermined portion of packaging material from said packaging material, which predetermined portion of said packaging material comprises a first edge of said predetermined portion of said packaging material and a second edge being disposed opposite said first edge of said predetermined portion of said packaging material;
folding said predetermined portion of said packaging material, disposing said first edge adjacent said second edge, and disposing said first edge and said second edge opposite a fold in said predetermined portion of said packaging material to prepare said folded predetermined portion of said packaging material to form a bag package;
welding a first side, a second side, and a third side connecting said first side and said second side of said folded predetermined portion of said packaging material and forming bag packages having an open portion between said first side and said second side and opposite said third side from said packaging material, which bag packages are thus configured to accept a product;
transporting said formed bag packages to be filled;
gripping the sides of said bag packages and holding open said open portions of said bag packages;
filling said bag packages with a product;
transporting said filled bag packages to be sealed;
welding the remaining open portion of said filled bag packages and thereby sealing said filled bag packages;
transporting said sealed, filled bag packages to be packaged;
packaging said sealed, filled bag packages into boxes; and
said step of treating said packaging material comprising treating said packaging material with sound to minimize the presence of living contaminants before said packaging material enters said aseptic clean room.

2. The method according to claim 1, wherein:
the packaging material is guided across at least one strip-like contact area that extends transversely across the path of the strip-format packaging material and forms the ultrasound head for the packaging material;
the packaging material is moved continuously across the ultrasound head;
the intensity of the ultrasound treatment can be modified by changing the angle of incidence of the ultrasound oscillations or the ultrasound energy onto the packaging material and/or by the selection of the geometry of the at least one ultrasound head and/or by the application force of the packaging material against the at least one ultrasound head;
the angle of incidence of the ultrasound head is adjustable;
a medium which promotes the ultrasound treatment is applied to the packaging material before and/or during the ultrasound treatment;
the medium is a liquid and/or a gas;
the medium that promotes the ultrasound treatment is a medium that improves the transmission of the ultrasound energy from the at least one ultrasound head to the packaging material;
the medium that promotes the ultrasound treatment is applied by means of the at least one ultrasound head to the packaging material;
the ultrasound treatment is performed by a plurality of ultrasound heads or ultrasound units and specifically simultaneously or chronologically delayed with respect to one another;
the ultrasound treatment is performed at a frequency in the range between approximately 20,000 Hertz and 1 Gigahertz, or in the range between approximately 20,000 Hertz and 35,000 Hertz; and
the ultrasound energy is controlled as a function of the speed at which the packaging material passes the at least one ultrasound head.

3. A means for performing the method according to claim 1 comprising:
means for unrolling a roll of packaging material and feeding said packaging material to an opening in an aseptic clean room;
means for feeding said packaging material under tension through said opening and means for simultaneously treating said packaging material as said packaging material moves through said opening;
means for cutting a predetermined portion of packaging material from said packaging material, which predetermined portion of said packaging material comprises a first edge of said predetermined portion of said packaging material and a second edge being disposed opposite said first edge of said predetermined portion of said packaging material;
means for folding said predetermined portion of said packaging material, disposing said first edge adjacent said second edge, and disposing said first edge and said second edge opposite a fold in said predetermined portion of said packaging material to prepare said folded predetermined portion of said packaging material to form a bag package;
means for welding a first side, a second side, and a third side connecting said first side and said second side of said folded predetermined portion of said packaging material and forming bag packages having an open portion between said first side and said second side and opposite said third side from said packaging material, which bag packages are thus configured to accept a product;
means for transporting said formed bag packages to be filled;
means for gripping the sides of said bag packages and holding open said open portions of said bag packages;
means for filling said bag packages with a product;
means for transporting said filled bag packages to be sealed;
means for welding the remaining open portion of said filled bag packages and thereby sealing said filled bag packages;
means for transporting said sealed, filled bag packages to be packaged;
means for packaging said sealed, filled bag packages into boxes; and
said means for treating said packaging material comprising means for treating said packaging material with sound to minimize the presence of living contaminants before said packaging material enters said aseptic clean room.

4. An arrangement for performing the method according to claim 1 comprising:
an unrolling arrangement being configured to unroll a roll of packaging material and feeding said packaging material to an opening in an aseptic clean room;
a feeding arrangement being configured to feed said packaging material under tension through said opening and a treating arrangement being configured to simultaneously treat said packaging material as said packaging material moves through said opening;
a cutting arrangement being configured to cut a predetermined portion of packaging material from said packaging material, which predetermined portion of said packaging material comprises a first edge of said predetermined portion of said packaging material and a second edge being disposed opposite said first edge of said predetermined portion of said packaging material;
a folding arrangement being configured to fold said predetermined portion of said packaging material, disposing said first edge adjacent said second edge, and disposing said first edge and said second edge opposite a fold in said predetermined portion of said packaging material to prepare said folded predetermined portion of said packaging material to form a bag package;
a welding arrangement being configured to weld a first side, a second side, and a third side connecting said first side and said second side of said folded predetermined portion of said packaging material and forming bag packages having an open portion between said first side and said second side and opposite said third side from said packaging material, which bag packages are thus configured to accept a product;
a transporting arrangement being configured to transport said formed bag packages to be filled;
a gripping arrangement being configured to grip the sides of said bag packages and holding open said open portions of said bag packages;
a filling arrangement being configured to fill said bag packages with a product;
a transporting arrangement being configured to transport said filled bag packages to be sealed;
a welding arrangement being configured to weld the remaining open portion of said filled bag packages and thereby sealing said filled bag packages;
a transporting arrangement being configured to transport said sealed, filled bag packages to be packaged;
a packaging arrangement being configured to package said sealed, filled bag packages into boxes; and
said treating arrangement being configured to treat said packaging material with sound to minimize the presence of living contaminants before said packaging material enters said aseptic clean room.

5. A method of aseptically treating, forming, filling, and closing packages in a packaging arrangement, said method comprising the steps of:
feeding a packaging material into a clean room and substantially simultaneously treating said packaging material;
removing a predetermined portion of packaging material from said packaging material;
forming, from said predetermined portion of said packaging material, an open package configured to accept a product;
filling said packages with a product;
closing and sealing said filled bag packages; and
said step of treating said packaging material comprising treating said packaging material with sound to minimize the presence of living contaminants in or on said packaging material.

6. The method according to claim 5, wherein:
said packaging material is guided across at least one strip-like contact area of an ultrasound head, which contact area extends transversely across the path of said packaging material; and
the intensity of the ultrasound treatment can be modified by changing the angle of incidence of the ultrasound oscillations or the ultrasound energy onto the packaging material and/or by the selection of the geometry of the at least one ultrasound head and/or by the application force of the packaging material against the at least one ultrasound head.

7. The method according to claim 6, wherein said packaging material is moved substantially continuously across the ultrasound head under mechanical tension.

8. The method according to claim 7, wherein the angle of incidence of the ultrasound head is adjustable.

9. The method according to claim 8, wherein a medium which promotes the ultrasound treatment is applied to the packaging material before and/or during the ultrasound treatment.

10. The method according to claim 9, wherein the medium is a liquid and/or a gas.

11. The method according to claim 10, wherein the medium that promotes the ultrasound treatment is a medium that improves the transmission of the ultrasound energy from the at least one ultrasound head to the packaging material.

12. The method according to claim 11, wherein:
the medium that promotes the ultrasound treatment is applied by means of the at least one ultrasound head to the packaging material; and
the ultrasound treatment is performed by a plurality of ultrasound heads or ultrasound units and specifically simultaneously or chronologically delayed with respect to one another.

13. The method according to claim 12, wherein:
the ultrasound treatment is performed at a frequency in the range between approximately 20,000 Hertz and 1 Gigahertz, or in the range between approximately 20,000 Hertz and 35,000 Hertz; and the ultrasound energy is controlled as a function of the speed at which the packaging material passes the at least one ultrasound head.

14. A means for performing the method according to claim 5 comprising:
feeding a packaging material into a clean room and substantially simultaneously treating said packaging material;
removing a predetermined portion of packaging material from said packaging material;
forming, from said predetermined portion of said packaging material, an open package configured to accept a product;
filling said packages with a product;
closing and sealing said filled bag packages; and
said step of treating said packaging material comprising treating said packaging material with sound to minimize the presence of living contaminants in or on said packaging material.

15. An arrangement for performing the method according to claim 5 comprising:
feeding a packaging material into a clean room and substantially simultaneously treating said packaging material;
removing a predetermined portion of packaging material from said packaging material;
forming, from said predetermined portion of said packaging material, an open package configured to accept a product;
filling said packages with a product;
closing and sealing said filled bag packages; and
said step of treating said packaging material comprising treating said packaging material with sound to minimize the presence of living contaminants in or on said packaging material.

16. A method for the sterilization of packaging material, in particular for the sterilization of films or similarly flexible wrapping materials by the irradiation of the packaging material (1) with mechanical ultrasound energy to kill microorganisms by exposure to the action of mechanical and/or thermal energy, wherein the packaging material (1) is moved under mechanical tension over at least at least one ultrasound head (6) of at least one ultrasound unit (3).

17. The method according to claim 16, wherein:
the packaging material (1) is guided across at least one strip-like contact area that extends transversely across the path of the strip-format packaging material (1) and forms the ultrasound head (6) for the packaging material;
the packaging material is moved continuously across the ultrasound head (6);
the intensity of the ultrasound treatment can be modified by changing the angle of incidence of the ultrasound oscillations or the ultrasound energy onto the packaging material (1) and/or by the selection of the geometry of the at least one ultrasound head (6) and/or by the application force of the packaging material against the at least one ultrasound head (6);
the angle of incidence of the ultrasound head (6) is adjustable;
a medium which promotes the ultrasound treatment is applied to the packaging material (1) before and/or during the ultrasound treatment; and
the medium is a liquid and/or a gas.

18. The method according to claim 17, wherein:
the medium that promotes the ultrasound treatment is a medium that improves the transmission of the ultrasound energy from the at least one ultrasound head (6) to the packaging material (1);
the medium that promotes the ultrasound treatment is applied by means of the at least one ultrasound head (6) to the packaging material (1);
the ultrasound treatment is performed by a plurality of ultrasound heads (6) or ultrasound units (3) and specifically simultaneously or chronologically delayed with respect to one another;
the ultrasound treatment is performed at a frequency in the range between approximately 20,000 Hertz and 1 Gigahertz, or in the range between approximately 20,000 Hertz and 35,000 Hertz; and
the ultrasound energy is controlled as a function of the speed at which the packaging material (1) passes the at least one ultrasound head (6).

19. A device for performing the method of claim 16, comprising at least one ultrasound system with at least one ultrasound unit (3) with an ultrasound head (6), over which the strip-format packaging material (1) is guided.

20. The device according to claim 19, wherein the at least one ultrasound (6) forms at least one strip-shaped contact surface for the packaging material (1) which runs transversely across the path of the packaging material (1).
